(19) United States
(12) Patent Application Publication
(10) Pub. No.: US 2009/0060978 A1
(43) Pub. Date: Mar. 5, 2009

(54) RESORBABLE BARRIER MICRO-MEMBRANES FOR ATTENUATION OF SCAR TISSUE DURING HEALING

(76) Inventor: Lukas Bluecher, Eurasburg (DE)

Correspondence Address: Kenton R. Mullins;Stout, Uxa, Buyan & Mullins, LLP, 4 Venture, Suite 300, Irvine, CA, 92618, US

(21) Appl. No.: 12/199,760

(22) Filed: Aug. 27, 2008

Related U.S. Application Data
(60) Provisional application No. 60/966,782, filed on Aug. 27, 2007.
(60) Provisional application No. 60/966,861, filed on Aug. 29, 2007.

(51) Int. Cl. A61K 9/70; A61K 38/18

(52) U.S. Cl. 424/426; 514/12

(57) ABSTRACT

Resorbable lactide polymer micro-membranes are disclosed. The micro-membranes are constructed of polylactide resorbable polymers, which are engineered to be absorbed into the body relatively slowly over time in order to reduce potential negative side effects. The membranes are formed to have very thin thicknesses, for example, thicknesses between about 0.010 mm and about O.300 mm. The membranes can be extruded from polylactide polymers having a relatively high viscosity property, can be preshaped with relatively thick portions, and can be stored in sterile packages.

### CROSS-REFERENCE TO RELATED APPLICATIONS

This application claims the benefit of U.S. Provisional Application No. 60/966,782, filed Aug. 27, 2007 (Att. Docket MB8039PR), and U.S. Provisional Application No. 60/966,861, filed Aug. 29, 2007 (Att. Docket MB8039PR2), the contents of both which are expressly incorporated herein by reference in their entirety. This application is related to U.S. application Ser. No. 10/385,399, filed Mar. 10, 2003 and entitled Resorbable Barrier Micro-Membranes for Attenuation of Scar Tissue During Healing (Att. Docket MA9496CON), now U.S. Pat. No. 6,673,362, the contents of which are expressly incorporated herein by reference in its entirety.

This application is also related to U.S. application Ser. No. 10/631,980, filed Jul. 31, 2003 (Att. Docket MA9604P), U.S. application Ser. No. 11/203,660, filed Aug. 12, 2005 (Att. Docket MB9828P), and, U.S. application Ser. No. 10/019,797, filed Jul. 26, 2002 (Att. Docket MB9962P). The foregoing applications are commonly assigned and the entire contents of all of them are expressly incorporated herein by reference in their entirety.

### BACKGROUND OF THE INVENTION

1. Field of the Invention

The present invention relates generally to resorbable membranes and methods of using the membranes, and of their use as medical implants.

2. Description of Related Art

A major clinical problem relating to surgical repair or inflammatory disease is adhesion which occurs during the initial phases of the healing process after surgery or disease. Adhesion is a condition which involves the formation of abnormal tissue linkages caused by the formation of fibrous scar tissue. These linkages can, for example, impair bodily function, produce infertility, obstruct the intestines and other portions of the gastrointestinal tract (e.g., bowel obstructions) and produce general discomfort, e.g. pelvic pain. The condition can in some instances be life threatening. The most common form of adhesion occurs as a result of surgical interventions, although adhesion may occur as a result of other processes or events such as pelvic inflammatory disease, mechanical injury, radiation treatment and the presence of foreign material.

Various attempts have been made to prevent postoperative adhesions. For example, the use of peritoneal lavage, heparinized solutions, procoagulants, modification of surgical techniques such as the use of microscopic or laparoscopic surgical techniques, the elimination of talc from surgical gloves, the use of smaller sutures and the use of physical barriers (membranes, gels or solutions) aiming to minimize apposition of serosal surfaces, have all been attempted. Unfortunately, limited success has been seen with these methods. Additionally, barrier materials in various forms, such as membranes and viscous intraperitoneal solutions designed to limit tissue apposition have also met with limited success. These barrier materials can include cellulosic barriers, polytetrafluoroethylene materials, and dextran solutions.

U.S. Pat. No. 5,795,584 to Tokahura et al. discloses anti-adhesion or scar tissue reduction films or membranes, and U.S. Pat. No. 6,136,333 to Cohn et al. discloses similar structures. In the Tokahura et al. patent, a bioabsorbable polymer is copolymerized with a suitable carbonate and then formed into a non-porous single layer adhesion barrier such as a film. In the Cohn et al. patent, a polymeric hydrogel for the prevention or attenuation of adhesion is formed without crosslinking by using urethane chemistry. Both of these patents involved relatively complex chemical formulas and/or reactions resulting in particular structures for use as surgical adhesion barriers. There continues to be a need to for an improved membrane to help minimize or prevent the formation of adhesions.

### SUMMARY OF THE INVENTION

The present invention provides an improved resorbable micro-membrane that can be used in various surgical contexts, for example, to inhibit, retard, or prevent tissue adhesions and reduce scarring. Furthermore, the co-polymers of the present invention may facilitate provision of relatively simple chemical reactions and/or formulations, and/or may facilitate provision of one or more of enhanced or more controllable mechanical strength and/or accelerated or more controllable degradation relative to other, e.g., mother, poly(esters).

In accordance with one feature of the present invention a resorbable micro-membrane is provided comprising, consisting essentially of, or consisting of a substantially uniform composition of a dual block copolymer. As embodied herein, the dual block copolymer can comprise a first block that may include, consist essentially of, or consist of a polylactide and/or a polyglycolide (e.g., (e.g., poly lactic acid (PLA), poly glycolic acid (PGA), or poly lactoglycolic acid (PLGA) and a second block that may include, consist essentially of, or consist of a polyethylene glycol (e.g., PEG). The first block, denoted as a PLA/PGA block, preferably may comprise a hydrophobic and biodegradable PLA/PGA block, and the second block, denoted as a PEG block, preferably may comprise a hydrophilic PEG block.

In accordance with another feature of the present invention, a resorbable micro-membrane is provided comprising consisting essentially of, or consisting of a substantially uniform composition of a tri block copolymer, which may comprise a first block that may include, consist essentially of, or consist of a polylactide and/or a polyglycolide (e.g., PLA, PGA, or PLGA), a second block that may include or consist of a polyethylene glycol (e.g., PEG), and a third block that may include or consist of a polylactide and/or a polyglycolide (e.g., PLA, PGA, or PLGA). The first and third blocks, each denoted as a PLA/PGA block, preferably may comprise hydrophobic and biodegradable PLA/PGA blocks, and the second block, denoted as a PEG block, preferably may comprise a hydrophilic PEG block.

The first PLA/PGA block and the second PEG block together may form a PLA/PGA-PEG copolymer, and addition of the third PLA/PGA block may result in the formation of a tripartite PLA/PGA-PEG-PLA/PGA copolymer. These PLA/PGA-PEG (and/or PLA/PGA-PEG-PLA/PGA) copolymer membranes can be formed, without limitation, by extrusion and can be made to possess, for example, an initial, relatively high viscosity (high viscosity property). The initial high viscosity property may facilitate reliable formation of the membrane by, for example, attenuating the occurrence of, for example, breaking or tearing of the membrane, during the extrusion process. After processing and sterilization, the viscosity of the polymer comprising membrane may typically be lower. Other viscosity properties (e.g., relatively high viscosity properties) can be used according to other aspects of the invention, in order, for example, to increase the strength of the PLA/PGA-PEG (and/or PLA/PGA-PEG-PLA/PGA) copolymer material during the extrusion process. In modified embodiments, the initial viscosity property may not be relatively high. The extrusion process may provides the membrane with a biased molecular orientation.

According to another feature of the invention, a membrane has a first substantially-smooth surface and a second substantially-smooth surface, is non-porous, and is about 0.01 mm to about 0.300 mm thick as measured between the first substantially-smooth surface and the second substantially-smooth surface. The membrane can comprise at least one relatively thick portion, which can form at least a segment of an edge of the membrane. The membrane thus can possess a varying cross-sectional thickness.

While the apparatus and method have or will be described herein for the sake of grammatical fluidity with functional explanations, it is to be expressly understood that the claims, unless expressly so indicated, are not to be construed as limited in any way by the construction of “means” or “steps” limitations, but are rather to be accorded the full scope of the meaning and equivalents of the claim language under the judicial doctrine of equivalents.

Any feature or combination of features described herein are included within the scope of the present invention provided that the features included in any such combination are not mutually inconsistent as will be apparent from the context, this specification, and the knowledge of one of ordinary skill in the art. In addition, any feature or combination of features may be specifically excluded from any embodiment of the present invention. For purposes of summarizing the present invention, certain aspects, advantages and novel features of the present invention are described. Of course, it is to be understood that not necessarily all such aspects, advantages or features will be embodied in any particular implementation of the present invention. Additional advantages and aspects of the present invention are apparent in the following detailed description and claims that follow.

### BRIEF DESCRIPTION OF THE DRAWINGS

FIG. 1a illustrates a micro-membrane, wherein a molecular orientation of the PLA/PGA-PEG (and/or PLA/PGA-PEG-PLA/PGA) copolymer is biased along an axis;

FIG. 1b illustrates a micro-membrane, wherein the molecular orientation of the PLA/PGA-PEG (and/or PLA/PGA-PEG-PLA/PGA) copolymer is biased along two axes;

FIG. 2a illustrates a micro-membrane having a thick portion;

FIG. 2b illustrates a micro-membrane having a thick portion forming a segment of an edge of the membrane;

FIG. 2c illustrates a micro-membrane having a thick portion forming an edge of the membrane;

FIGS. 2d and 2e illustrate a membrane having more than one thick portion thereon.

FIG. 2f illustrates a membrane having thick portions with holes;

FIG. 3a illustrates a laminotomy procedure wherein a portion of the posterior arch (lamina) of a vertebra is surgically removed;

FIG. 3b is an enlarged view of FIG. 3a;

FIG. 3c illustrates a micro-membrane for application to the exiting nerve root of the spinal chord in accordance with a first pre-formed embodiment of the present invention;

FIG. 4 illustrates a micro-membrane for application to two exiting nerve roots of the spinal chord in accordance with a second pre-formed embodiment of the present invention; and

FIG. 5 illustrates a micro-membrane for application to four exiting nerve roots of the spinal chord in accordance with a third pre-formed embodiment of the present invention.

### DETAILED DESCRIPTION OF THE PRESENTLY PREFERRED EMBODIMENTS

Reference will now be made in detail to the presently preferred embodiments of the invention, examples of which are illustrated in the accompanying drawings. Wherever possible, the same or similar reference numbers are used in the drawings and the description to refer to the same or like parts. It should be noted that the drawings are in simplified form and are not drafted to precise scale. In reference to the disclosure herein, for purposes of convenience and clarity only, directional terms, such as, top, bottom, left, right, up, down, over, above, below, beneath, rear, and front, are used with respect to the accompanying drawings. Such directional terms should not be construed to limit the scope of the invention in any manner unless expressly so indicated in the claims.

Although the disclosure herein refers to certain illustrated embodiments, it is to be understood that these embodiments are presented by way of example and not by way of limitation. The intent of this disclosure, while discussing exemplary embodiments, is that the following detailed description be construed to cover all modifications, alternatives, and equivalents of the embodiments as may fall within the spirit and scope of the invention as defined by the appended claims.

Barrier membranes of the present invention may be constructed from various biodegradable materials, such as resorbable polymers. In accordance with one embodiment, non-limiting polymers which may be used to form barrier membranes of the present invention include a dual block copolymer. As embodied herein, the dual block copolymer can comprise a first block that may include, consist essentially of, or consist of a polylactide and/or a polyglycolide (e.g. poly lactic acid (PLA), poly glycolic acid (PGA), or poly lactoglycolic acid (PLGA) and a second block that may include, consist essentially of, or consist of a polyethylene glycol (e.g., PEG). The first block, denoted as a PLA/PGA block, preferably may comprise a hydrophobic and biodegradable PLA/PGA block, and the second block, denoted as a PEG (polyethylene glycol) block, preferably may comprise a hydrophilic PEG block.

In accordance with another feature of the present invention, a resorbable micro-membrane is provided comprising consisting essentially of, or consisting of a substantially uniform composition of a tri block copolymer, which may comprise a first block that may include, consist essentially of, or consist of a polylactide and/or a polyglycolide (e.g., PLA, PGA, or PLGA), a second block that may include or consist of a polyethylene glycol (e.g., PEG), and a third block that may include or consist of a polylactide and/or a polyglycolide (e.g., PLA, PGA, or PLGA). The first and third blocks, each denoted as a PLA/PGA block, preferably may comprise hydrophobic and biodegradable PLA/PGA blocks, and the second block, denoted as a PEG block, preferably may comprise a hydrophilic PEG block.

The first PLA/PGA block and the second PEG block together may form a PLA/PGA-PEG copolymer, and addition of the third PLA/PGA block may result in the formation of a tripartite PLA/PGA-PEG-PLA/PGA copolymer. These PLA/PGA-PEG (and/or PLA/PGA-PEG-PLA/PGA) copolymer membranes can be formed, without limitation, by extrusion and can be made to possess, for example, an initial, relatively high viscosity (high viscosity property) according to certain, but not all, implementations and aspects of the invention. The initial high viscosity property may in some instances facilitate reliable formation of the membrane by, for example, attenuating the occurrence of, for example, breaking or tearing of the membrane, during the extrusion process. After processing and sterilization, the viscosity of the polymer comprising membrane may typically be lower. Other viscosity properties (e.g., relatively high viscosity properties) and/or other viscosity properties, can be used according to other aspects of the invention. Some of these uses may be provided in order, for example, to increase the strength of the PLA/PGA-PEG (and/or PLA/PGA-PEG-PLA/PGA) copolymer material during the extrusion process. The extrusion process may provide the membrane with a biased molecular orientation.

In a presently preferred embodiment, the micro-membranes can be manufactured using extrusion procedures, such as for example those known in the art. The extrusion procedures advantageously can provide for efficient production of the membranes. Moreover, membranes which are manufactured by such extrusion techniques can be rendered free from solvent trappings in the membrane and, furthermore, can be provided with, for example, a molecular bias, including a predetermined molecular bias along one or more axis. Monoaxial extrusion may be employed to manufacture the membranes in a preferred embodiment of the present invention. In a modified embodiment, biaxial extrusion procedures may be implemented to manufacture the membranes.

Thus, compositions of dual block PLA/PGA-PEG copolymer, and/or tri-block PLA/PGA-PEG-PLA/PGA copolymer, can be extruded to form the membranes of the present invention. In certain embodiments, PLA/PGA-PEG copolymers may take the form of one or more of the following polymers:
1. Poly(L-lactide-co-PEG),
2. Poly(L-lactide-co-DL-lactide-co-PEG), and
3. Poly(L-lactide-co-glycolide-co-PEG),
   and PLA/PGA-PEG-PLA/PGA copolymers may take the form of one or more of the following polymers:
4. Poly(L-lactide-co-PEG-co-L-lactide),
5. Poly(L-lactide-co-PEG-co-L-lactide-co-DL-lactide),
6. Poly(L-lactide-co-PEG-co-L-lactide-co-glycolide),
7. Poly(L-lactide-co-DL-lactide-co-PEG-co-L-lactide-co-DL-lactide),
8. Poly(L-lactide-co-DL-lactide-co-PEG-co-L-lactide-co-glycolide), and
9. Poly(L-lactide-co-glycolide-co-PEG-co-L-lactide-co-glycolide),

Such polymers can be synthesized de novo, or may be purchased from, without limitation, Boehringer Ingelheim KG of Germany, for formulation into the present compositions and extrusion into the membranes of the present invention.

Exemplary chemical structures, and synthesis and nomenclature conventions to be used herein follow, wherein:

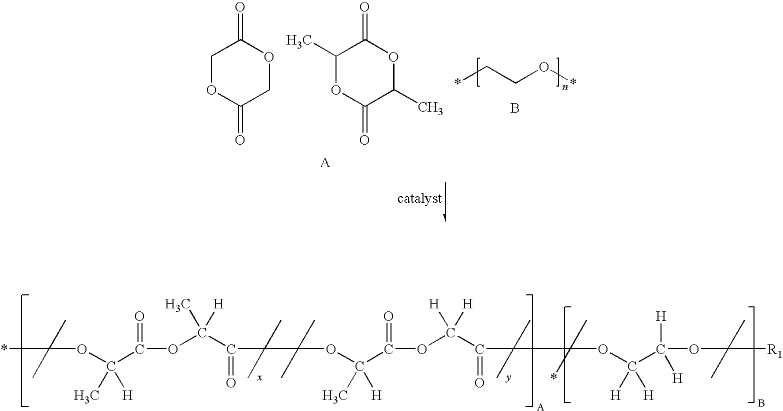

The immediately preceding diagram shows the formation of an exemplary block polymer A from random ring-opening co-polymerization of two different monomers: the cyclic 1,4-dioxane-2,5-diones of glycolic acid and lactic acid. Common catalysts include tin(II) 2-ethylhexanoate, tin(II) alkoxides, or aluminum isopropoxide. The resulting block A comprises a random co-polymerization of glycolate and lactate monomers into a lactate-co-poly glycolate polymer.

PLGA degrades by hydrolysis of its ester linkages in the presence of water. It has been shown that the time required for degradation of PLGA is related to the monomers' ratio used in production: the higher the content of glycolide units, the lower the time required for degradation. An exception to this rule is the copolymer with 50:50 monomers' ratio which exhibits the faster degradation (about two months). In addition, polymers that are end-capped with esters (as opposed to the free carboxylic acid) demonstrate longer degradation half-lives. PLGA has been successful as a biodegradable polymer because it undergoes hydrolysis in the body to produce the monomers lactic acid and glycolic acid. These two monomers under normal physiological conditions are by-products of various metabolic pathways in the body. Since the body effectively deals with the two monomers, there is very minimal systemic toxicity associated with using PLGA for drug delivery or biomaterial applications.

Scheme B shows the incorporation of polyethylene glycol (PEG) units into the block co-polymer with PLGA, again by the action of the catalyst. PEG also has low systemic toxicity, and is currently used in various medical and pharmaceutical agents.

The resulting block co-polymer can be represented schematically as follows:

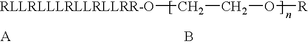

### Nomenclature:

Commercially obtained PLGA:PEG block co-polymers include the RESOMER® PEG products from Boeringer Ingelheim.
A particularly preferred (though non-exclusive) product is RESOMER® PEG Sample MD Type LRP d 70 5 5, wherein LR stands for RESOMER Acronym LR (A-block), P stands for PEG (B-block), 70 stands for the mole ratio within the A-block, the first 5 stands for the weight percent PEG, and the second 5 stands for the molecular weight of the PEG divided by one thousand.

Typical examples of PLA/PGA-PEG (and/or PLA/PGA-PEG-PLA/PGA) copolymers are as follows: For controlled release (CR), the polymer will usually contain from about 5% to about 15% PEG. For medical devices (MD) the polymer will usually contain less than about 5% PEG. For controlled release the A block may contain e.g., D,L-lactide-co-glycolide (RG). For Medical Devices, the A Block may contain e.g., L lactide (L), L-lactide-co-D,L-lactide (LR), or L Lactide-co-glycolide (LG).

In accordance with one aspect of the present invention, the membranes have a particular range of a viscosity property. As used herein, a “viscosity property” is a measure of the viscosity of a polymeric dilute solution viscosity, expressed as the ratio of the natural logarithm of the relative viscosity to the concentration of the polymer in grams per 100 milliliters of solvent. Viscosity property may be understood by persons of skilled in the art to be the inherent viscosity of a solution at a given temperature, as conventionally used in the art. In one embodiment, the membranes of the present invention have a molecular bias, indicating they were formed from an extrusion technique, and a relatively high viscosity property. In other embodiments, the membranes of the present invention may have molecular biases coupled with viscosities that are not relatively high, such as medium-ranged viscosity properties or relatively-low viscosity properties.

In accordance with one aspect of the present invention, it is discovered that a copolymer composition having a pre-extrusion viscosity property greater than about 5 g/dL can be extruded to form the relatively thin membranes (for example, about 0.01 mm to about 0.300 mm) of the present invention. Other relatively high viscosity properties, such as those above 4 g/dL can be used according to other aspects of the invention, in order to assure sufficient strength, or to increase the strength, of the copolymer composition material during for example the extrusion process. In modified embodiments, relatively-low initial viscosities may range from about 0.7 to about 0.95 dl/g.

The initially high (i.e., pre-extrusion) viscosity property of the PLA/PGA-PEG (and/or PLA/PGA-PEG-PLA/PGA) copolymer may facilitate reliable and reproducible formation of the membrane by for example attenuating an occurrence of weakening, breaking or tearing of the membrane, during the extrusion process. Although after processing and sterilizing the viscosity property of the membrane is lower that this initial high value, the initial relatively high viscosity property of the membrane can, in some implementations, advantageously contribute to or help to facilitate reliably reproducible extrusions down to a thickness on the order of fractions of a millimeter. For example it is discovered that a PLA/PGA-PEG (and/or PLA/PGA-PEG-PLA/PGA) copolymer having an initial (pre-extrusion) viscosity property may be extruded to form a membrane, having a thickness of about 0.02 mm. The use of sterilizing techniques will not significantly change the viscosity property of this material. In one embodiment, ethylene oxide is used as an agent for sterilizing the membranes (which may comprise “micro-membranes”). It is believed that ethylene oxide does not cause a substantial reduction in the viscosity property of the micro-membranes whereby, according to use of such agent(s) (and/or other agent(s) having a similar and/or a less dramatic effect than electron-beam use on the viscosity property during sterilization) for sterilization, the post-sterilization viscosity properties may remain about the same as their pre-sterilization viscosity properties, or may be reduced by a factor of, for example, about 5% to about 15%, or may be reduced by a factor of, for example, about 20% to about 50%. In embodiments wherein other sterilizing techniques are used, such as electron-beam sterilization, the resulting viscosity property may be reduced by about 1.25 g/dL to about 1.75 g/dL. In embodiments other implementations wherein techniques such as electron-beam sterilization is used, the extruded membranes can have viscosity properties greater than about 1 g/dL. In one embodiment, the membranes have a viscosity property greater than about 2 g/dL. Furthermore, other non-equivalent, modified implementations may comprise a post-sterilization viscosity property that is less than about 1 g/dL, or less than about 0.9 g/dL, or less than about 0.7 g/dL, or even less than about 0.5 g/dL.

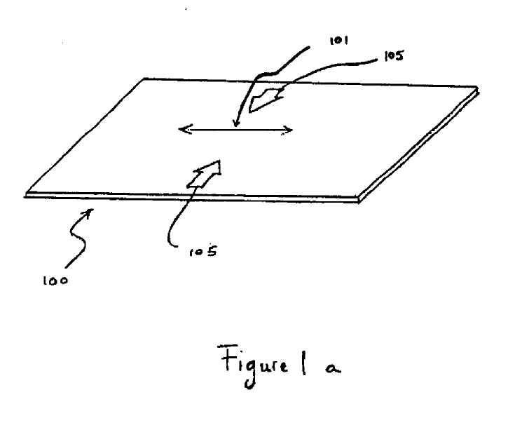

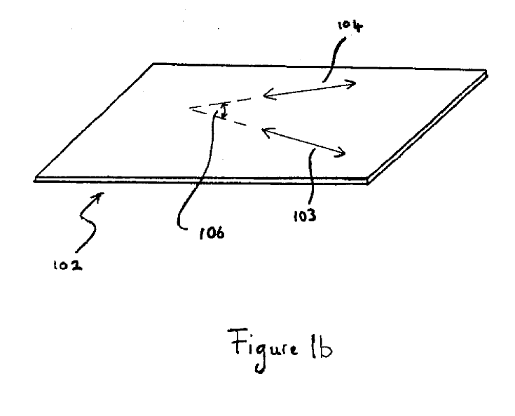

In accordance with one aspect of the invention, the molecular orientations of the PLA/PGA-PEG (and/or PLA/PGA-PEG-PLA/PGA) copolymers can be biased. The above-discussed extrusion process can provide such biased molecular orientations. The biased molecular orientations may be predetermined so that a suitable process, such as a suitable extrusion process, may be employed in the manufacture of the membranes disclosed herein. In one embodiment, polymer chains of the membrane are substantially aligned on one axis, as shown in FIG. 1a. For example, in this embodiment more than about 65% and preferably more than about 80% of the polymer chains or segments of polymer chains are aligned on an axis 101 of a micro-membrane 100.

In one embodiment, the polymer chains are substantially aligned on two axes. FIG. 1b shows a membrane 102 having both a first axis 103 and a second axis 104 on which the polymers are aligned. In such an embodiment, more than about 50%, preferably more than about 90%, of the polymer chains or segments of polymer chains are substantially aligned on one of the two axes. In one embodiment, the aligned polymers are substantially equally proportioned between the first axis 103 and the second axis 104. In another embodiment, the aligned polymers lie more on one axis than the other axis. For example, the aligned polymers may be aligned more along the first axis 103 than the second axis 104. For example, the polymers can be about 45% aligned on the first axis and about 55% aligned on the second axis. In one embodiment, the axes form an angle 106 of less than 80 degrees. Preferably, the axes form an angle 106 of less than about 45 degrees, more preferably less than 30 degrees, and even more preferably less than 20 degrees.

The molecular orientations of the PLA/PGA-PEG (and/or PLA/PGA-PEG-PLA/PGA) copolymers can confer various physical characteristics to the membrane. For example, when subjected to heat treatment sufficient to bring the membrane to its glass transition temperature, a membrane having a biased molecular orientation may shrink in the direction substantially perpendicular to an axis. As shown in FIG. 1a, when the membrane 100 having a biased molecular orientation is subjected to heat treatment, the direction of shrinkage 105 may be substantially perpendicular to the axis 101. In addition, the biased molecular orientation may permit the direction of shrinkage to be controlled or selectively controlled when the membrane is heated. This may be advantageous in situations where specific configurations and sizes are desired for the implantation of the membranes.

In one embodiment, during an extrusion process a membrane is output through an extrusion head or orifice having a first thickness and, subsequently, the membrane is stretched down to a second thickness, wherein the first thickness is greater than the second thickness. The first thickness can be greater than two times the second thickness, more preferably greater than five times the second thickness, and more preferably greater than ten times the second thickness. Accordingly, when the processed and sterilized membrane is subsequently brought to its glass transition temperature (e.g., which may range for instance from about 40 to 60 degrees Celsius, and in a one example may have a value of about 45 degrees Celsius and in another example may have a value of about 55 degrees Celsius ), its thickness will or may return back to the first thickness.

In one embodiment, a membrane of the present invention does not shrink uniformly in all directions when it is subjected to heat treatment. Preferably, a membrane of the present invention shrinks substantially in a direction perpendicular to the molecular orientation axis or axes, and does not substantially shrink in the direction parallel to the molecular orientation axis, when brought to a glass transition temperature of the membrane. For example, a membrane of the present invention may shrink about 5% to about 30% in the direction perpendicular to the molecular orientation axis or axes, and may shrink about 1% to about 5% in the direction parallel to the molecular orientation axis. In one example, when the processed and sterilized membrane is subsequently brought to its glass transition temperature, it will shrink in a direction substantially perpendicular to the alignment axis (e.g., 101) or axes (e.g., 103, 104) in an amount approximately proportional to the amount it was stretched in the initial extrusion process. As presently embodied, the shrinkage in the direction perpendicular to the alignment axis or axes will continue until a thickness of the membrane returns from the second thickness to the first thickness.

A membrane of the present invention can have at least one substantially smooth surface. Preferably, a membrane of the present invention has two (opposing) substantially smooth surfaces. As measured between the opposing surfaces, a membrane of the present membrane can have a thickness of about 0.01 mm to about 0.3 mm and, more preferably, about 0.0 1 mm to about 0. 1 mm. In a preferred embodiment, a membrane of the present invention has a thickness of about 0.015 mm to about 0.025 mm. In another preferred embodiment, a membrane of the present invention has a thickness of about 0.02 mm.

A membrane of the present invention may further comprise at least one thick portion protruding from at least one of the two substantially smooth surfaces. In a preferred embodiment, the at least one thick portion protrudes from both of the two substantially smooth surfaces. In other words, the membrane may include multiple regions or portions with different thicknesses. In one embodiment, the membrane includes a first portion having a first thickness, and a second portion having a second thickness, where the first thickness is greater than the second thickness. The first portion may be located away from an edge of the membrane, or the first portion may be located at the edge of the membrane. In addition, the first portion has a length which is not greater than the length or width of the membrane. In certain embodiments, the length is shorter than both the length and the width of the membrane.

The output orifice of the extruding device may have a shape which corresponds to a cross section of the membrane. For example, in order to generate a membrane having relatively thick portions on two opposing edges of the membrane, the output orifice of the extruding device may comprise a generally rectangular shape having a width and a height, wherein the shape is modified by the height of the output orifice being greater at the two opposing edges of the output orifice than in an area between the two opposing edges of the output orifice. In such a configuration, the profile of heights across the width of the output orifice roughly corresponds to the profile of thicknesses across the width of the micro-membrane. In other embodiments, a micro-membrane having for example thick portions on opposing edges thereof may be generated using an extrusion device having a rectangular output orifice. In other embodiments, the thick portion(s) may be formed by means, such as machining, which can be implemented alone or in combination with, for example, the above-discussed extrusion processes. In addition to the above processes which can generate monoaxial molecular alignments, wherein for example, about 80% or more of the membrane molecular alignment is in one direction, a membrane having a biaxial molecular orientation may be generated using for example a circular output orifice wherein pressurized air is blown into a tubular, micro-membrane outputted through the circular output orifice.

Preferably, the thick portion is effective to provide an attachment function to the membrane. In modified embodiments, the thick portion may be effective to provide rigidity to at least a portion of the membrane. In one embodiment, each thick portion has a length equal to or less than a length of the membrane, a width of about 0.5 mm to about 25 mm (and in one embodiment not wider than the width of the membrane), and a thickness of about 2 to about 10 times thicker than the thickness of the membrane.

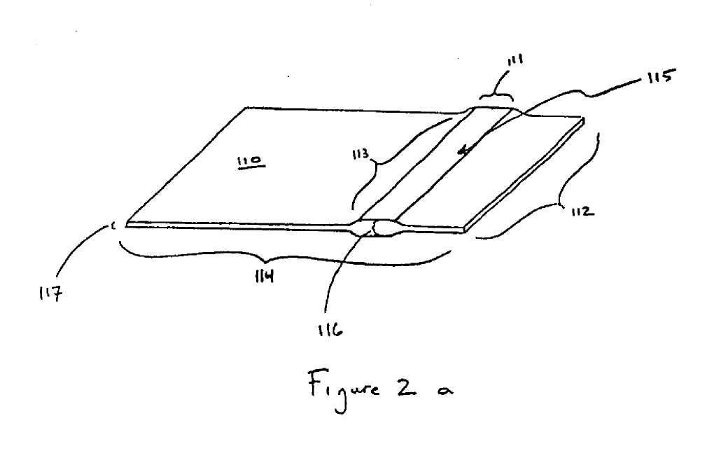

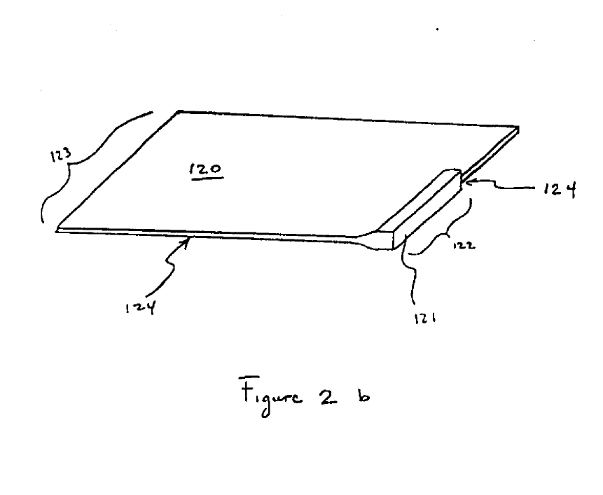

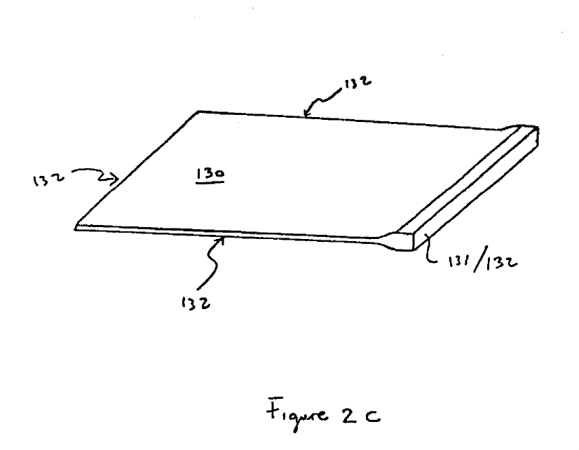

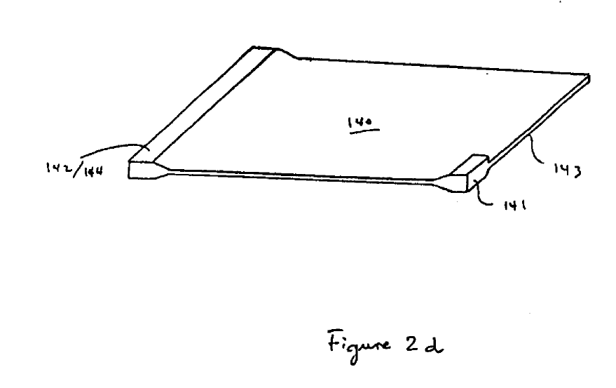

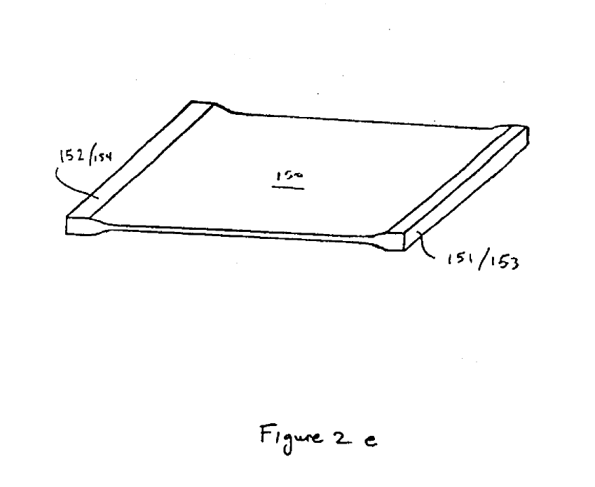

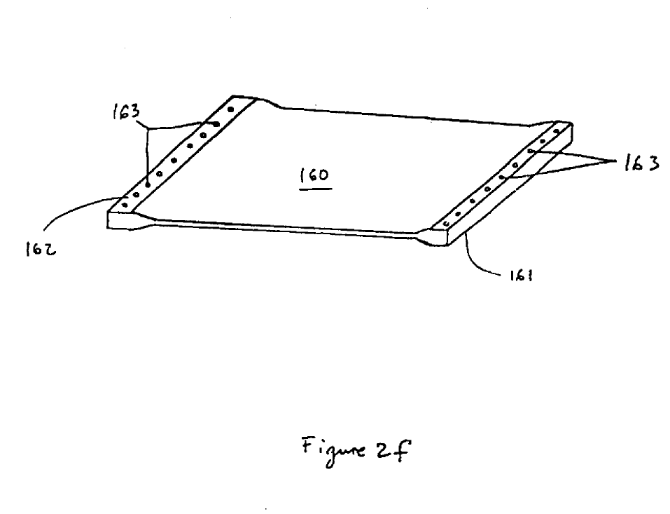

For example, FIG. 2a shows a thick membrane portion 115. In this figure, a length 113 of the thick portion 115 is equal to that of the length of the membrane 112, a width of the thick portion 111 is shorter than the width of the membrane 114, and a thickness of the thick portion 116 is about three times the thickness of the membrane 117. In view of the disclosure herein, thick portion 115 corresponds to a first portion having a first thickness, and the remaining portions of the illustrated membrane 112 correspond to a second portion having a second thickness which is less than the first thickness.

In one embodiment, the thick portion has a length shorter than that of the membrane. For example, FIG. 2b shows a membrane 120 comprising a thick portion 121. The length of the thick portion 122 is smaller than the length of the membrane 123. The length of the thick portion 122 is also smaller than the width of the membrane 123. In one embodiment, the thick portion may form a segment of an edge of the membrane or may form the entire edge of the membrane. For example, FIG. 2b shows a thick portion forming a segment of an edge of a membrane 124. FIG. 2c shows a membrane 130 having four edges 132, one of which is formed by a thick portion 131. In one embodiment, a membrane comprises more than one thick portion. For example, FIG. 2d shows a membrane 140 having a first thick portion 141 forming a segment of the first edge 143, and a second thick portion 142 forming a segment of the second edge 144.

A preferred micro-membrane of the present invention can comprise a substantially uniform composition of PLA/PGA-PEG (and/or PLA/PGA-PEG-PLA/PGA) copolymer. The PLA/PGA-PEG (and/or PLA/PGA-PEG-PLA/PGA) copolymer can have a biased molecular orientation in the membrane as a consequence, for example, of extrusion. Furthermore, the membrane can comprises a first and a second thick portion, each thick portion having a width of about 5 mm to about 25 mm and a thickness of about 0.070 mm. The micro-membrane can have a thickness of about 0.02 mm as measured between the surfaces. FIG. 2e shows such an embodiment of a membrane 150 having a first thick portion 151 forming a first edge 153, and a second thick portion 152 forming a second edge 154. Additional thick portions may be formed on additional edges or areas of the membrane 124 in other modified embodiments. For example, four thick portions may be formed on four corresponding edges of a rectangular membrane.

A membrane of the present invention may further comprise a plurality of holes disposed along at least one edge of the membrane. Preferably, these holes extend through the membrane. In one embodiment, the edges having the holes may be formed by at least one thick portion. For example, FIG. 2f shows a membrane 160 having a first thick portion 161 and a second thick portion 162. The thick portions have holes 163 along their lengths. The holes may facilitate for example, suturing of the micro-membrane to tissue.

On the topic of attachment, various means for attaching the micro-membrane to structures such as muscular tissue, other soft tissue, or bone are contemplated, and these means may be used with or without holes. According to a preferred embodiment, however, the attachment means are implemented on the actual thick portions of the micro-membrane, although this is not required. In addition to sutures, staples may be used to attach the membrane to, for example, soft tissue such as, in an exemplary instance, the paravertebral muscle. As another example, the membrane and/or the bridging membrane (described infra) may be secured to, for example, hard tissue such as, in an exemplary instance, the vertebrae bone, using resorbable bone screws or tacks. Tucking or folding the membrane material into anatomical crevices may be sufficient to fix either membrane's position in certain instances. An adhesive such as a fibrin sealant, or a resorbable cyanoacrylate adhesive may further or alternatively be utilized to secure one or more of the membranes, alone or in combination with the above means of attachment. In an exemplary embodiment, the above attachment protocols may be applied to the thick portions.

Each thick portion on the membrane may have a width for example of about 0.5 mm to about 25 mm. In one embodiment, the thick portion may have a width of about 5 to about 25 mm, which may be useful for suturing purposes. In another embodiment, the thick portion may have a width of about 0.5 mm, which may be useful for heat bonding as described below.

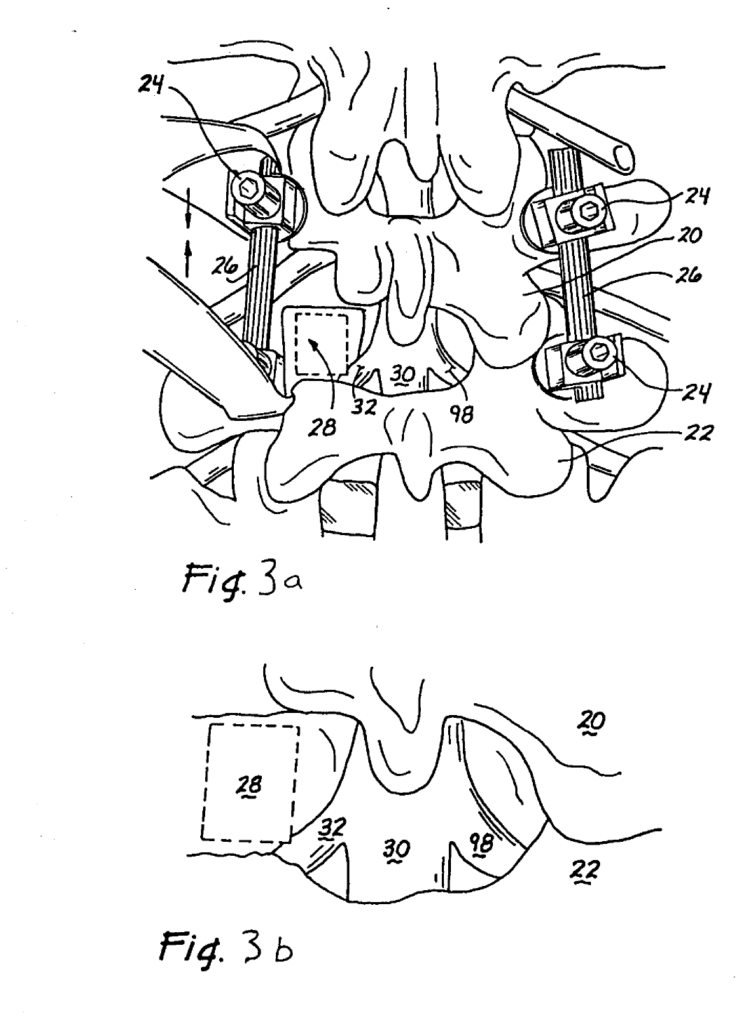

In accordance with one aspect of the present invention, a thick portion can be heat bonded, such as with a bipolar electro-cautery device, ultrasonically welded, or similarly sealed directly to tissue, such as soft tissue, which may comprise, in an exemplary instance the dura of a spinal chord 30 and an exiting nerve root 32 (FIG. 3a). Such a device can be used to heat the membrane at various locations in addition to the thick portions, such as at non-thick edges and at points in the middle, to at least above a glass transition temperature of the membrane, and preferably above its softening point temperature. The material can be heated along with adjacent tissue such that the two components bond together at their interface. In another embodiment, the thick portions or other areas of the membrane can be heat bonded or sealed directly to one or both of two target sites, such as in an exemplary instance two vertebrae 20 and 22 (FIG. 3a), or to muscle or other soft tissue, for example. In yet another embodiment, the thick portions or other areas of the micro-membrane can be heat bonded or sealed directly to itself in an application, for example, wherein the membrane is wrapped around a structure and then heat joined to itself. Moreover, the technique of heat-sealing the membrane to itself or body tissue may be combined with another attachment method for enhanced anchoring. For example, the micro-membrane material may be temporarily affixed in position using two or more points of heat sealing (i.e., heat welding) using an electro-cautery device, and sutures, staples or glue can then be added to secure the micro-membrane into place.

The micro-membrane of the present invention may be more effective than other membranes in accordance with embodiments in which it is very smooth and non-porous. For example, a lack of porosity can operate to form a barrier that does not allow interaction of the tissues. The non-porosity and the smoothness of the exemplary embodiment of a micro-membrane can reduce tissue turbulence, enhance tissue guidance, and/or minimize scar formation. Moreover, the smooth, uninterrupted surface of the exemplary micro-membrane embodiment may facilitate movement of tissue (e.g., the dura) and/or other local tissues (e.g., across the area), hence reducing, for example, frictional rubbing and wearing which may induce scar tissue formation.

As used herein, the term “non-porous” refers to a material which is generally watertight and, in accordance with a preferred embodiment, not fluid permeable. However, in a modified embodiment of the invention micro-pores (which are i.e., fluid permeable but not cell permeable) may exist in the micro-membrane of the present invention, to the extent, for example, that they do not substantially disrupt the smoothness of the surfaces of the resorbable micro-membrane to cause scarring of tissue. In substantially modified embodiments for certain applications, pores which are cell permeable but not vessel permeable may be manufactured and used.

As presently embodied, many of the thinner membrane thicknesses can be sufficiently contoured even in the absence of heating to glass transition temperature. In one embodiment, membranes of the present invention can be capable of resorbing (i.e., being absorbed by the mammalian body) within a period, for example, of about 10 to 20 weeks, or of about 20 to 30 weeks, or, according to other implementations, up to about 18 months, or up to about 24 months from an initial implantation of the membrane into the mammalian body. The micro-membranes may be used in a number of surgical applications, including: surgical repair of fracture orbital floors, surgical repair of the nasal septum and perforated ear drum micro-membrane, as a protective sheathing to facilitate osteogenesis, surgical repair of the urethral anatomy and repair of urethral strictures, prevention of synostosis in completed corrective surgery for cranial fusions and forearm fractures, lessening of soft-tissue fibrosis or bony growth, as a temporary covering for prenatal rupture omphalocele during staged repair procedures, guided tissue regeneration between the teeth and gingival margin, tympanic membrane repairs, dural coverings and neural repair, heart vessel repair, hernia repair, tendon anastomoses, temporary joint spacers, wound dressings, scar coverings, and as a covering for gastroschisis. The micro-membrane of the present invention can be particularly suitable for preventing tissue from abnormally fibrotically joining together following surgery, which can lead to abnormal scarring and/or interfere with normal physiological functioning. In some cases, such scarring can force and/or interfere with follow-up, corrective, or other surgical operations.

For example, there is evidence pointing to epidural adhesions as possible factors contributing to failed surgical procedures, such as failed back surgery. For instance, epidural fibrosis may occur following spinal injuries or as a post-operative surgical complication. The dense scar formation which may occur, for example, on dura and around nerve roots, has previously been described as the “laminotomy membrane,” and has been implicated in rendering subsequent spine operations technically more difficult. In a laminectomy procedure, for example, the micro-membrane of the present invention can desirably be inserted between the dural sleeve and the paravertebral musculature post laminotomy and can conform readily to block exposed marrow elements of the laminae. Imposition of the membrane material as a barrier between the paravertebral musculature and the epidural space is believed to reduce cellular trafficking and vascular invasion into the epidural space from the overlying muscle and adjacent exposed cancellous bone. Moreover, the present micro-membrane may avoid interference with normal posterior wound healing while at the same time inhibiting the unwanted adhesions and scarring.

The very thin construction of these membranes is believed to substantially accelerate the rate of absorption of the membranes, compared to rates of absorption of thicker membrane implants of the same material. It is believed, however, that resorption into the body too quickly of the membrane may, in some instances, yield undesirable drops in local pH levels, thus introducing/elevating, for example, local inflammation, discomfort and/or foreign antibody responses. Further, a resulting uneven (e.g., cracked, broken, roughened or flaked) surface of a micro-membrane degrading too early may undesirably cause tissue turbulence between the tissues before, for example, adequate healing has occurred, potentially resulting in tissue inflammation and/or scarring.

In other instances, a different (e.g., more rapid) resorption may be desired in one or more areas of a patient, and/or at one or more points in time of one or more surgical procedures, so that, in accordance with an aspect of the present invention, rates of absorption may be controlled or varied, temporally and/or spatially, by varying the materials of the membrane or parts thereof.

Micro-membranes in accordance with an aspect of the present invention may be provided in rectangular shapes that are for example several centimeters on each side, or can be cut and formed into other specific shapes, configurations and sizes, by the manufacturer before packaging and sterilization. In modified embodiments, various known formulations and copolymers of, for example, polylactides may affect the physical properties of the micro-membrane and/or the bridging membrane (described infra). The micro-membranes of the present invention may be sufficiently flexible to conform over and/or around anatomical structures, although some heating in a hot water bath may be necessary for thicker configurations. In modified embodiments, certain polylactides which may become somewhat more rigid and brittle at thicknesses above, for example, 0.25 mm and which may be softened by formation with other polymers, copolymers and/or other monomers, e.g., epsilon-caprolactone, for example, may be implemented to form micro-membranes.

Moreover, in accordance with another aspect of the present invention, the micro-membrane and/or the bridging membrane (described infra) may comprises a substance for cellular control, such as at least one of a chemotactic substance for influencing cell-migration, an inhibitory substance for influencing cell-migration, a mitogenic growth factor for influencing cell proliferation and a growth factor for influencing cell differentiation. Such substances may be impregnated in the membrane, but may also be coated on one or more surfaces of the membrane. In addition, substances may be contained in discrete units on or in the membrane, which may be effective to facilitate selective release of the substances when the membrane is inserted into a patient.

FIG. 3a illustrates a laminotomy procedure wherein two vertebrae 20 and 22 are separated and fixated using screws 24 and rods 26, and a portion of the lamina has been removed, leaving a window 28 (shown as a phantom rectangle) in the vertebrae 22. FIG. 3b is an enlarged view of the window 28 in the lamina of the vertebrae 22. The spinal chord 30 and an exiting nerve root 32 are thus exposed. In accordance with an implementation of the present invention, the micro-membrane is applied to the dura of both the spinal chord 30 and the exiting nerve root 32, to thereby attenuate or eliminate the occurrence of post-operative scarring in the vicinity of the exiting nerve root 32.

In a modified embodiment, a thicker bridging membrane can also be applied to one or both of the vertebrae 20 and 22, to thereby bridge (i.e., tent) over and cover the window 28. This bridging membrane may be non-porous, fluid permeable, cell permeable or vessel permeable in accordance with various embodiments, and preferably comprises a thickness between about 0.5 mm and 2.0 mm for preventing prolapse of adjacent muscle tissue into the foramen (i.e., the spinal lumen containing the spinal chord 30 and exiting nerve root 32). In accordance with various embodiments, the bridging membrane may be used alone or in combination with the scar-reduction resorbable barrier micro-membrane, or the scar-reduction resorbable barrier membrane may be used without the bridging membrane.

In other embodiments, the bridging membrane disclosed herein may take on any one or more characteristics or functions, in any combination, of the tissue-ingrowth biodegradable region described in U.S. application Ser. No. 11/203,660, with the thin membrane disclosed herein taking on any one or more characteristics or functions, in any combination, of the tissue-ingrowth biodegradable region described in U.S. application Ser. No. 11/203,660.

Turning to FIG. 3c, a pre-formed micro-membrane 34 can be formed with a first welding flange 36 and a second welding flange 38 thereon. The welding flanges can be constructed to be thick portions or to have thick portions merely along their edges. Moreover, in modified embodiments thick portions may be formed on other edges of the below-described membranes, on other portions of the membranes, and/or any combinations thereof. A trunk portion 40 can be formed to fit over a first anatomical structure, such as the spinal chord 30, and a branch portion 42 can be formed to fit over a second anatomical structure, such as the exiting nerve root 32. The first welding flange 36 can be formed by a first slit 44 and a second slit 46, and the second welding flange 38 can be formed by a first slit 48 and a second slit 50. In application, the pre-formed micro-membrane 34 can be placed over one or more structures, such as the spinal chord 30 and the exiting nerve root 32, and, subsequently, the first welding flange 36 and the second welding flange 38 can be positioned (e.g., bent) at least partially in proximity to, over and/or around the structure or structures (e.g., exiting nerve root). The rounded end 52 of the branch portion 42 can, for example, be placed onto a portion of the exiting nerve root 32 furthest away from the spinal chord 30. As presently embodied, the first welding flange 36 and the second welding flange, in the current example, can be wrapped around, and preferably tucked beneath (i.e., behind) the exiting nerve root 32. In accordance with a typical implementation, the first welding flange 36 can be heat welded to the second welding flange 38. The flanges can be cut, according to certain implementations, to wrap entirely around a structure, such as the exiting nerve root 32, and, for example, to overlap one another. The first welding flange 36 may be sutured to the second welding flange 38, alone or in addition with the heat welding step, to thereby secure the first welding flange 36 to the second welding flange 38. In another embodiment, neither heat welding nor suturing are used and the flanges are merely tucked partially or completely around one or more structures, such as the exiting nerve root 32 (e.g., depending on the dimensions of the root 32). When sutures are to be used, the pre-formed micro-membrane 34 may be pre-formed and packaged with, for example, optional suture apertures 60. The edges 64 and 66 can then be heat welded to the structure (e.g., the spinal chord 30). The two edges 68 and 70 can form a third welding flange 72. A fourth welding flange 74 can be formed by slits 76 and 78, and a fifth welding flange 80 can be formed by slits 82 and 84. The welding flanges may be secured in manners similar to those discussed in connection with the welding flanges 36 and 38. Heat welds may further be secured along other edges and along the surface of the pre-formed micro-membrane 34. Moreover, notches may be formed on the membranes of the present invention, such as, for example, at the ends 64 and 66 in modified-shape embodiments, for accommodating, for example, the spinal processes.

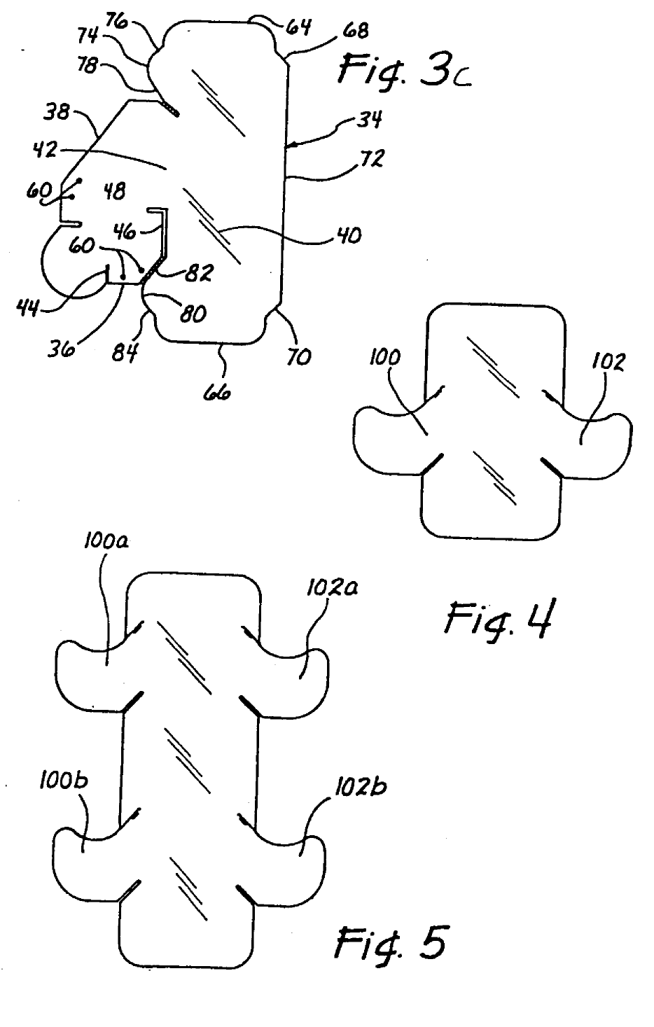

FIG. 4 illustrates a micro-membrane for application, for example, to two exiting nerve roots 32 and 98 of the spinal chord in accordance with another pre-formed embodiment of the present invention. FIG. 5 illustrates a micro-membrane similar to that of FIG. 4 but adapted for application, for example, to four exiting nerve roots of the spinal chord in accordance with another pre-formed embodiment of the present invention. For example, the branch portion 100 may be analogized in structure and operation to the branch portion 42 of the FIG. 3 embodiment, and the other branch portion 102 can be constructed to accommodate, for example, the exiting nerve root 98. Similar elements are shown in FIG. 5 at 100a, 102a, 100b and 102c.

Other configurations for accommodating different anatomical structures may be formed. For example, configurations may be designed to be formed into, for example, cone structures to fit around base portions with protrusions extending through the centers of the membranes. Suture perforations may be formed around perimeters of the membranes, and cell and vessel permeable pores may be included as well.

In general, any particulars, features or combinations thereof (in whole or in part, in structure or step), described or referenced herein, may be combined with any particulars, features or combinations thereof (in whole or in part, in structure or step), described or referenced in any of the documents mentioned herein, including without limitation U.S. application Ser. No. 11/203,660 (in whole or in part, in structure or step, provided that the particulars or features included in any such combination are not mutually inconsistent.

In accordance with one implementation of the present invention, the pre-formed micro-membranes can be preformed and sealed in sterilized packages for subsequent use by the surgeon. Since one objective of the micro-membranes of the present invention can be to reduce sharp edges and surfaces, preformation of the membranes is believed to help, in some instances, facilitate, albeit to a relatively small degree, rounding of the edges for less rubbing, tissue turbulence and inflammation. That is, the surfaces and any sharp edges of the micro-membranes are believed to be capable of ever so slightly potentially degrading over time in response to exposure of the membranes to moisture in the air, to thereby form rounder edges. This is believed to be an extremely minor effect. Moreover, any initial heating to glass temperature of the pre-cut membranes just before implanting may conceivably further round any sharp edges. Furthermore, the very micro-membranes of the present invention may be particularly susceptible, at least theoretically, to these phenomena, and, perhaps to a more noticeable extent, are susceptible to tearing or damage from handling, thus rendering the pre-forming of the micro-membranes potentially beneficial for preserving the integrity thereof.

In accordance with an aspect of the present invention, a surgical prosthesis (e.g., a resorbable scar-tissue reduction micro-membrane system) can comprise an adhesion-resistant region (e.g., a biodegradable region, a biodegradable side, a membrane and/or a micro-membrane) of PLA/PGA-PEG (and/or PLA/PGA-PEG-PLA/PGA) copolymer as described herein, and further may comprise an optional tissue-ingrowth region (e.g., another membrane, a bridging membrane, a biodegradable region and/or a biodegradable side or mesh) which may or may not comprise, for example, a PLA/PGA-PEG (and/or PLA/PGA-PEG-PLA/PGA) copolymer.

The surgical prosthesis (e.g., biodegradable surgical prosthesis) can be constructed for use in the repair of soft tissue defects, such as soft tissue defects resulting from incisional and other hernias and soft tissue defects resulting from extirpative tumor surgery. The surgical prosthesis may also be used in cancer surgeries, such as surgeries involving sarcoma of the extremities where saving a limb is a goal. Other applications of the surgical prosthesis of the present invention may include laparoscopic or standard hernia repair in the groin area, umbilical hernia repair, paracolostomy hernia repair, femora hernia repair, lumbar hernia repair, and the repair of other abdominal wall defects, thoracic wall defects and diaphragmatic hernias and defects.

According to an aspect of the present invention, the tissue-ingrowth region and the adhesion-resistant region may differ in both (A) surface appearance and (B) surface function. For example, the tissue-ingrowth region can be constructed with at least one of a surface topography (appearance) and a surface composition (function), either of which may facilitate strength, longevity or lack thereof, and/or a substantial fibroblastic reaction in the host tissue relative to for example the anti-adhesion region. On the other hand, the adhesion-resistant region can be constructed with at least one of a surface topography and a surface composition, either of which may facilitate, relative to the tissue-ingrowth region, an anti-adhesive effect between the biodegradable surgical implant and host tissues.

### A. Surface Topography (Appearance):

The tissue-ingrowth region can be formed to have an open, non-smooth and/or featured surface comprising, for example, alveoli and/or pores distributed regularly or irregularly. In further embodiments, the tissue-ingrowth region can be formed to have, additionally or alternatively, an uneven (e.g., cracked, broken, roughened or flaked) surface which, as with the above-described surfaces, may cause tissue turbulence (e.g., potential tissue inflammation and/or scarring) between host tissues and the tissue-ingrowth region.

Over time, with respect to the tissue-ingrowth region, the patient's fibrous and collagenous tissue may substantially completely overgrow the tissue-ingrowth region, growing over and affixing the tissue-ingrowth region to the tissue. In one implementation, the tissue-ingrowth region comprises a plurality of alveoli or apertures visible to the naked eye, through or over which the host tissue can grow and achieve substantial fixation.

As an example, pores may be formed into the tissue-ingrowth region by punching or otherwise machining, or by using laser energy. Non-smooth surfaces may be formed, for example, by abrading the tissue-ingrowth region with a relatively course surface (e.g., having a 40 or, preferably, higher grit sandpaper-like surface) or, alternatively, non-smooth surfaces may be generated by bringing the tissue-ingrowth region up to its softening or melting temperature and imprinting it with a template (to use the same example, a sandpaper-like surface). The imprinting may occur, for example, during an initial formation process or at a subsequent time.

On the other hand, the adhesion-resistant region can be formed to have a closed, continuous, smooth and/or non-porous surface. In an illustrative embodiment, at least a portion of the adhesion-resistant region is smooth comprising no protuberances, alveoli or vessel-permeable pores, so as to attenuate occurrences of adhesions between the tissue-ingrowth region and host tissues.

In a molding embodiment, one side of the press may be formed to generate any of the tissue-ingrowth region surfaces discussed above and the other side of the press may be formed to generate an adhesion-resistant region surface as discussed above. Additional features (e.g., roughening or forming apertures) may subsequently be added to further define the surface of, for example, the tissue-ingrowth region. In an extrusion embodiment, one side of the output orifice may be formed (e.g. ribbed) to generate a tissue-ingrowth region (wherein subsequent processing can further define the surface such as by adding transverse ribs/features and/or alveoli) and the other side of the orifice may be formed to generate an adhesion-resistant biodegradation region surface. In one embodiment, the adhesion-resistant region is extruded to have a smooth surface and in another embodiment the adhesion-resistant region is further processed (e.g., smoothed) after being extruded.

### B. Surface Composition (Function):

As presently embodied, the tissue-ingrowth region comprises a first material, and the adhesion-resistant region comprises a second material which is different from the first material. In modified embodiments, the tissue-ingrowth region and the adhesion-resistant region may comprise the same or substantially the same materials. In other embodiments, the tissue-ingrowth region and the adhesion-resistant region may comprise different materials resulting from, for example, an additive having been introduced to at least one of the tissue-ingrowth region and the adhesion-resistant region.

According to an implementation of the present invention, the adhesion-resistant region is constructed to minimize an occurrence of adhesions of host tissues (e.g., internal body viscera) to the surgical prosthesis. In modified embodiments, the adhesion-resistant region and the tissue-ingrowth region of the surgical prosthesis may be formed of the same material or relatively less divergent materials, functionally speaking, and the adhesion-resistant region may be used in conjunction with an anti-inflammatory gel agent applied, for example, onto the adhesion-resistant region at a time of implantation of the surgical prosthesis. According to other broad embodiments, the adhesion-resistant region and the tissue-ingrowth region may be formed of any materials or combinations of materials disclosed herein (including embodiments wherein the two regions share the same layer of material) or their substantial equivalents, and the adhesion-resistant region may be used in conjunction with an anti-inflammatory gel agent applied, for example, onto the adhesion-resistant region at a time of implantation of the surgical prosthesis.

The tissue-ingrowth region can be formed of similar and/or different materials to those set forth above, to facilitate strength, longevity or lack thereof, and/or direct post-surgical cell colonization via, for example, invoking a substantial fibroblastic reaction in the host tissue. In an illustrated embodiment, the tissue-ingrowth region is constructed to be substantially incorporated into the host tissue and/or to substantially increases the structural integrity of the surgical prosthesis. Following implantation of the surgical prosthesis, body tissues (e.g., subcutaneous tissue and/or the exterior fascia) commence to incorporate themselves into the tissue-ingrowth region. While not wishing to be limited, it is believed that the body, upon sensing the presence of the tissue-ingrowth region of the present invention, is disposed to send out fibrous tissue which grows in, around and/or through and at least partially entwines itself with the tissue-ingrowth region. In this manner, the surgical prosthesis can become securely attached to the host body tissue.

Regarding different materials, according to an aspect of the present invention, the tissue-ingrowth region can comprises a (e.g., resorbable) polymer composition having one or more different characteristics than that or those of a (e.g., resorbable) polymer composition of the adhesion-resistant region. The different characteristics may include (1a) time or rate of biodegradation affected by additives, (1b) time or rate of biodegradation affected by polymer structures/compositions, (2) polymer composition affecting strength or structural integrity, and (3) ability to facilitate fibroblastic reaction.

In accordance with a method of the present invention, the surgical prosthesis can be used to facilitate repair of, for example, a hernia in the ventral region of a body. An implanted surgical prosthesis having both an adhesion-resistant region disposed on one side and having a tissue-ingrowth region disposed on a second side of the surgical prosthesis can be provided. The abdominal wall can include muscle enclosed and held in place by an exterior fascia and an interior fascia. An interior layer, called the peritoneum, can cover the interior side of the interior fascia. The peritoneum is a softer, more pliable layer of tissue that forms a sack-like enclosure for the intestines and other internal viscera. A layer of skin and a layer of subcutaneous fat cover the exterior fascia.

Surgical repair of a soft tissue defect (e.g., a hernia) can be performed by using, for example, conventional techniques or advanced laparoscopic methods to close substantially all of a soft tissue defect. According to one implementation, an incision can be made through the skin and subcutaneous fat, after which the skin and fat can be peeled back followed by any protruding internal viscera (not shown) being positioned internal to the hernia. In certain implementations, an incision can be made in the peritoneum followed by insertion of the surgical prosthesis into the hernia opening so that the surgical prosthesis is centrally located in the hernia opening. One or both the tissue-ingrowth region and the adhesion-resistant region may be attached by, e.g., suturing to the same layer of the abdominal wall, e.g., the relatively-strong exterior fascia. Alternatively, the adhesion-resistant region may be attached to another member, such as the interior fascia and/or the peritoneum. The tissue-ingrowth region can be surgically attached to the exterior fascia while the adhesion-resistant region can be attached to the tissue-ingrowth region and/or optionally to the exterior fascia using, e.g., heat bonding, suturing, and/or other affixation protocols disclosed herein or their substantial equivalents. Those possessing skill in the art will recognize that other methods of sizing/modifying/orientating/attaching a surgical prosthesis of this invention may be implemented according to the context of the particular surgical procedure.

The size of the surgical prosthesis typically will be determined by the size of the defect. Use of the surgical prosthesis in a tension-free closure may be associated with less pain and less incidence of post surgical fluid accumulation. Exemplary sutures may be implemented to at least partially secure the surgical prosthesis to the abdominal wall structure. The sutures can be implemented so that no lateral tension is exerted on the exterior fascia and/or muscle. When disrupted, the skin and fat may be returned to their normal positions, with for example the incisional edges of the skin and fat being secured to one another using suitable means such as subsurface sutures.

In modified embodiments of the present invention, one or both of the tissue-ingrowth region and the adhesion-resistant region of the surgical prosthesis, can be heat bonded (or in a modified embodiment, otherwise attached, such as by suturing). Heat bonding may be achieved, for example, with a bipolar electro-cautery device, ultrasonicly welding, or similar sealing between the tissue-ingrowth region and the adhesion-resistant region and/or directly to surrounding tissues. Such a device can be used to heat the surgical prosthesis at various locations, such as at edges and/or at points in the middle, at least above its glass transition temperature, and preferably above its softening point temperature. The material is heated, e.g., along with adjacent tissue, such that the two components bond together at their interface. The heat bonding may also be used initially, for example, to secure the tissue-ingrowth region to the adhesion-resistant region. Since the tissue-ingrowth region serves more of a load-bearing function, a few typical embodiments may exclude heat-bonding as the sole means for securing this region to host tissues. In other embodiments, the technique of heat bonding the surgical prosthesis to itself or body tissue may be combined with another attachment method for enhanced anchoring. For example, the surgical prosthesis may be temporarily affixed in position using two or more points of heat bonding using an electro-cautery device, and sutures, staples or glue can subsequently (or in other embodiments, alternatively) be added to secure the surgical prosthesis into place.

The tissue-ingrowth region and the adhesion-resistant region may be arranged to form more than one layer or substantially one layer, or the regions may both belong to a single, integrally formed layer. For example, the tissue-ingrowth region and the opposing adhesion-resistant region may be arranged in two layers, wherein one of the regions is disposed on top of, and opposite to, the other region.

In one embodiment, the tissue-ingrowth region and the adhesion-resistant region may be combined on a single side of the surgical prosthesis in, for example, substantially one layer, wherein the regions are adjacent each other on one side of the surgical prosthesis. As a slight deviation, a surgical prosthesis having a tissue-ingrowth region on at least one (and preferably, both) side(s) thereof may be manufactured using any of the techniques described herein and, subsequently, an adhesion-resistant region may be formed on, e.g., one side, by smoothing, filling, or otherwise processing an area of the tissue-ingrowth region with a suitable material as disclosed herein or technique (e.g., coating or filling with a liquid or flowable polymer composition, and/or mechanically smoothing) to thereby form an adhesion-resistant region having adhesion-resistant properties relative to those of the tissue-ingrowth region.

Similarly, a patch comprising an adhesion-resistant region may be sized and affixed (e.g., heat bonded, such as with a bipolar electro-cautery device, ultrasonicly welded, or similarly affixed) at a time of implantation directly to at least one of the tissue-ingrowth region and surrounding host tissues. In modified embodiments, the affixing may be accomplished using, for example, press or adhesive bonding, or sutures. In further embodiments, at least part of the affixing may occur at a time of manufacture of the surgical prosthesis before packaging. The patch of adhesion-resistant region alternatively may be partially affixed (e.g., using techniques enumerated in this paragraph) at, for example, a non-perimeter or central area thereof to an area (e.g., a non-perimeter or central area) of the tissue-ingrowth region, so that a surgeon can trim the adhesion-resistant region (and/or the tissue-ingrowth region) at a time of implantation while the adhesion-resistant implant is affixed to the tissue-ingrowth region. For instance, a tissue-ingrowth region may substantially surround an adhesion-resistant region on one side of the surgical prosthesis, and only a tissue-ingrowth region may be formed on the other side of the surgical prosthesis. In such an implementation, the adhesion-resistant region of the surgical prosthesis can be sized and shaped so as to substantially cover any opening created by the soft tissue defect, with the tissue-ingrowth regions facilitating surgical attachment to, and incorporation into, the host tissue on at least one side of, and, preferably, on both sides of, the surgical prosthesis.

In modified embodiments, the tissue-ingrowth region and/or the adhesion-resistant region on a given surface or surfaces of the surgical prosthesis each may be of any size or shape suited to fit the particular soft tissue defect. For example, either of the tissue-ingrowth region and/or the adhesion-resistant region on a given surface of the surgical prosthesis may have shapes of ovals, rectangles and various complex or other shapes wherein, for each such implementation, the two regions may have essentially the same, or different, proportions and/or dimensions relative to one another.

In general, various techniques may be employed to produce the surgical prosthesis, which typically has one or two layers defining the tissue-ingrowth region and the adhesion-resistant region. Useful techniques include solvent evaporation methods, phase separation methods, interfacial methods, extrusion methods, molding methods, injection molding methods, heat press methods and the like as known to those skilled in the art. The tissue-ingrowth region and the adhesion-resistant region may comprise two distinct layers or may be integrally formed together as one layer.

The tissue-ingrowth region and the adhesion-resistant region may be partially or substantially entirely formed or joined together. Joining can be achieved by mechanical methods, such as by suturing or by the use of metal clips, for example, hemoclips, or by other methods, such as chemical or heat bonding.

The above-described embodiments have been provided by way of example, and the present invention is not limited to these examples. Multiple variations and modification to the disclosed embodiments will occur, to the extent not mutually exclusive, to those skilled in the art upon consideration of the foregoing description. Additionally, other combinations, omissions, substitutions and modifications will be apparent to the skilled artisan in view of the disclosure herein. As iterated above, any feature or combination of features described and referenced herein are included within the scope of the present invention provided that the features included in any such combination are not mutually inconsistent as will be apparent from the context, this specification, and the knowledge of one of ordinary skill in the art. For example, any of the implants and implant components, sub-components, or uses, and any particulars or features thereof, or other features, including method steps and techniques, may be used with any other structure and process described or referenced herein, in whole or in part, in any combination or permutation. Accordingly, the present invention is not intended to be limited by the disclosed embodiments, but is to be defined by reference to the appended claims.

What is claimed is:

1. A resorbable micro-membrane system for attenuating or preventing a formation of post-surgical scar tissue between a healing post-surgical site and adjacent surrounding tissue following an in vivo surgical procedure on the post-surgical site, the system having a pre-implant configuration immediately before the system is formed between the post-surgical site and the adjacent surrounding tissue, the system comprising:
a substantially planar membrane of resorbable polymer base material having a first substantially-smooth side and a second substantially-smooth side, the substantially planar membrane of resorbable polymer base material comprising a single layer of resorbable polymer base material between the first substantially-smooth side and the second substantially-smooth side, the single layer of resorbable polymer base material having a substantially uniform composition;
wherein a thickness of the single layer of resorbable polymer base material, measured between the first substantially-smooth side and the second substantially-smooth side, is between about 0.01 mm and about 0.300 mm;
wherein the single layer of resorbable polymer base material is non-porous; and
wherein the single layer of resorbable polymer base material is adapted to maintain a smooth-surfaced barrier between the healing post-surgical site and the adjacent surrounding tissue for a relatively extended period of time sufficient to attenuate or eliminate any formation of scar tissue between the post-surgical site and the adjacent surrounding tissue.
wherein the single layer of resorbable polymer base material comprises one or more of (a) a dual block copolymer including a first hydrophobic block of one or more of a lactide and a glycolide and a second hydrophilic block of a polyethylene glycol (PEG), and (b) a tri block copolymer including a first hydrophobic block of one or more of a lactide and a glycolide, a second hydrophilic block of a polyethylene glycol (PEG), and a third hydrophobic block of one or more of a lactide and a glycolide, wherein the third hydrophobic block is either the same or different from the first hydrophobic block.

2. The resorbable scar-tissue reduction micro-membrane system as set forth in claim 1, wherein the single layer of resorbable polymer base material comprises a dual block copolymer including a first hydrophobic block of one or more of a lactide and a glycolide and a second hydrophilic block of a polyethylene glycol.

3. The resorbable scar-tissue reduction micro-membrane system as set forth in claim 2, wherein the resorbable scar-tissue reduction micro-membrane system further includes another membrane, which comprises a thickness less than 2000 microns and which is permeable.

4. The resorbable scar-tissue reduction micro-membrane system as set forth in claim 1, wherein the single layer of resorbable polymer base material comprises a tri block copolymer including a first hydrophobic block of one or more of a lactide and a glycolide, a second hydrophilic block of a polyethylene glycol, and a third hydrophobic block of one or more of a lactide and a glycolide, wherein the third hydrophobic block is either the same or different from the first hydrophobic block.

5. The resorbable scar-tissue reduction micro-membrane system as set forth in claim 4, wherein the resorbable scar-tissue reduction micro-membrane system further includes another membrane, which comprises a thickness less than 2000 microns and which is permeable.

6. The resorbable scar-tissue reduction micro-membrane system as set forth in claim 1, wherein the single layer of resorbable polymer base material comprises a plurality of holes disposed along an edge of the single layer of resorbable polymer base material.

7. The resorbable scar-tissue reduction micro-membrane system as set forth in claim 6, wherein the single layer of resorbable polymer base material does not comprise any holes substantially away from the edge of the single layer of resorbable polymer base material.

8. The resorbable scar-tissue reduction micro-membrane system as set forth in claim 7, wherein the edge extends around the single layer of resorbable polymer base material.

9. The resorbable scar-tissue reduction micro-membrane system as set forth in claim 7, wherein a slit is formed in a periphery of the single layer of resorbable polymer base material so that the edge extends along the slit.

10. The resorbable scar-tissue reduction micro-membrane system as set forth in claim 6, wherein:
the single layer of resorbable polymer base material further comprises a plurality of holes disposed away from the edge;
each of the holes near the periphery has a first diameter;
each of the holes near the center has a second diameter; and
the first diameters are greater than the second diameters.

11. The resorbable scar-tissue reduction micro-membrane system as set forth in claim 1, wherein the thickness is about 100 microns.

12. The resorbable scar-tissue reduction micro-membrane system as set forth in claim 1, wherein the thickness is about 200 microns.

13. The resorbable scar-tissue reduction micro-membrane system as set forth in claim 1, wherein the single layer of resorbable polymer base material is not fluid permeable.

14. The resorbable scar-tissue reduction micro-membrane system as set forth in claim 1, wherein the single layer of resorbable polymer base material is impregnated with at least one of a chemotactic substance for influencing cell-migration, an inhibitory substance for influencing cell-migration, a mitogenic growth factor for influencing cell proliferation, a growth factor for influencing cell differentiation, and factors which promote neoangiogenesis.

15. The resorbable scar-tissue reduction micro-membrane system as set forth in claim 14, wherein the resorbable scar-tissue reduction micro-membrane system is sealed in a sterile packaging.

16 . The resorbable scar-tissue reduction micro-membrane system as set forth in claim 1, wherein the resorbable scar-tissue reduction micro-membrane system further includes another membrane, which comprises a thickness less than 2000 microns and which is permeable.

17. The resorbable scar-tissue reduction micro-membrane system as set forth in claim 16, wherein the other membrane is a bridging membrane.

18. The resorbable scar-tissue reduction micro-membrane system as set forth in claim 16, wherein the other membrane is fluid permeable.

19. The resorbable scar-tissue reduction micro-membrane system as set forth in claim 16, wherein the other membrane is cell permeable.

20. The resorbable scar-tissue reduction micro-membrane system as set forth in claim 16, wherein the other membrane is vessel permeable.

21. The resorbable scar-tissue reduction micro-membrane system as set forth in claim 16, wherein the other membrane comprises a thickness between 500 microns and 2000 microns.
